(19) United States
(12) Patent Application Publication
(10) Pub. No.: US 2024/0285979 A1
(43) Pub. Date: Aug. 29, 2024

(54) APPARATUS AND METHOD FOR IMPLEMENTING AXIAL FOCAL REGION TEMPERATURE MEASUREMENT DISTRIBUTION DETECTION THROUGH LATERAL PROBE

(71) Applicant: NANJING KHONS MEDTECH CO., LTD., Nanjing, Jiangsu (CN)

(72) Inventors: Xiangqing KONG, Nanjing (CN); Enguang ZHANG, Nanjing (CN); Ruibiao GUO, Nanjing (CN); Feifei CHE, Nanjing (CN); Jing CAI, Nanjing (CN)

(73) Assignee: NANJING KHONS MEDTECH CO., LTD., Nanjing, Jiangsu (CN)

(21) Appl. No.: 18/580,241

(22) PCT Filed: Aug. 21, 2023
(86) PCT No.: PCT/CN2023/113989

§ 371 Date: Jan. 18, 2024

(30) Foreign Application Priority Data
Nov. 2, 2022 (CN) 202211359322.6

(51) Int. Cl. A61N 7/02; G01K 11/22

(52) U.S. Cl. CPC A61N 7/02; G01K 11/22

(57) ABSTRACT

An apparatus and a method are disclosed for implementing axial focal region temperature measurement distribution detection through a lateral probe. The apparatus includes a temperature measurement module, an imaging ultrasound module, a lateral imaging probe, a ranging module, and a position control module. The imaging ultrasound module sends an electrical pulse signal to the lateral imaging probe, and obtains an ultrasound image and ultrasound radio frequency data through the lateral imaging probe; an axial of the lateral imaging probe always intersects with an axis of a focused ultrasound transducer; the temperature measurement module controls the lateral imaging probe to perform cyclic scanning on a temperature measurement ROI region through the position control module; and the ranging module is configured to obtain a mapping depth of focus D of the temperature measurement ROI region within a field of view of the lateral imaging probe.

### TECHNICAL FIELD

The present invention relates to an apparatus and a method for implementing axial focal region temperature measurement distribution detection through a lateral probe, and belongs to the field of biomedical instruments and devices.

### BACKGROUND

Focused ultrasound is a technology that implements non-invasive treatment by emitting an ultrasonic wave from outside of a body into the body and converging the ultrasonic wave into a focal region at a specific position, thereby producing a thermal effect in the focal region and nearby tissues.

Since the focused ultrasound mainly plays a therapeutic role by heating tissues, temperature measurement of the focal region and the nearby tissues is beneficial to an implementation of the focused ultrasound technology. In addition, temperature measurement is also a basis for further ultrasound dose control of the focused ultrasound. In addition, for a treatment target region with important or sensitive organs nearby, measurement of a temperature of the focal region and the nearby tissues also provides a means to ensure the safety of a focused ultrasound implementation process.

Patent ZL201710876349.5 publishes an ultrasound method for measuring temperature changes of biological tissues based on thermal expansion and a gating algorithm, which may be used for implementing temperature measurement of the focal region and the nearby tissues. However, due to limitation of designs of common focused ultrasound devices, the method is affected by artifacts in an actual scenario, which causes a temperature measurement failure. At present, a common focused ultrasound device on the market that uses imaging ultrasound for guidance generally includes an imaging ultrasound probe and a transducer apparatus for emitting focused ultrasound. The imaging ultrasonic probe is embedded in a central opening of the transducer. This structure design causes the imaging ultrasound probe to be interfered by artifacts caused by reflection between structures during imaging, and consequently, valid ultrasound radio frequency data cannot be outputted for temperature measurement calculation.

Therefore, an apparatus and a method that can resolve interference of artifacts are needed, to successfully apply the foregoing ultrasound temperature measurement method in an actual engineering device.

### SUMMARY

An objective of the present invention is to provide an apparatus and a method for implementing axial focal region temperature measurement distribution detection through a lateral probe, which can resolve a problem that an ultrasound temperature method cannot be applied since ultrasound radio frequency data is interfered with by artifacts caused by a coaxial design of an imaging ultrasound probe and a transducer, and can implement two-dimensional or three-dimensional temperature distribution detection in an axial focus region.

To achieve the foregoing objective, the present invention adopts the following technical solutions:

On one hand, the present invention provides an apparatus for implementing axial focal region temperature measurement distribution detection through a lateral probe, including a temperature measurement module, an imaging ultrasound module, a lateral imaging probe, a ranging module, and a position control module, where
the imaging ultrasound module is connected to the temperature measurement module and the lateral imaging probe, and the imaging ultrasound module sends an electrical pulse signal to the lateral imaging probe, and obtains an ultrasound image and ultrasound radio frequency data through the lateral imaging probe;
the lateral imaging probe is mounted on the position control module, an axis of the lateral imaging probe always intersects with an axis of a focused ultrasound transducer, and the position control module drives the lateral imaging probe to move, to cause an imaging field of view of the lateral imaging probe to traverse a temperature measurement ROI region through movement;
the position control module is connected to the temperature measurement module, and the temperature measurement module controls the lateral imaging probe to perform cyclic scanning on the temperature measurement ROI region through the position control module; and
the ranging module is connected to the temperature measurement module, and the ranging module is configured to obtain a mapping depth of focus D of the temperature measurement ROI region within the field of view of the lateral imaging probe, and transmit the mapping depth of focus D to the temperature measurement module.

Further, the ranging module includes a ranging processing unit and a capacitance-grid electronic rangefinder, and is configured to measure a distance from an intersection point of the axis of the focused ultrasound transducer and the axis of the lateral imaging probe to a foremost end surface of the lateral imaging probe, that is, the mapping depth of focus D, a distance compensation value is stored inside the ranging processor unit, and the distance compensation value is a distance between the axis of the focused ultrasound transducer and a front end surface of the lateral imaging probe when the lateral imaging probe is in an initial state.

Further, the position control module includes a parallel moving mechanism, and the parallel moving mechanism drives the lateral imaging probe to move upward and downward in an axis direction of the focused ultrasound transducer through a bracket. Based on the structural form, method steps for the ranging module to obtain the mapping depth of focus D include:
(1) measuring a movement distance of the lateral imaging probe along the axis of the lateral imaging probe through the capacitance-grid electronic rangefinder; and
(2) adding the movement distance and the distance compensation value together to obtain the mapping depth of focus D.

Alternatively, the position control module includes a fan-shaped rotating mechanism, and the fan-shaped rotating mechanism is arranged at a tail end of a bracket on which the lateral imaging probe is mounted, and drives a front end of the lateral imaging probe to perform fan-shaped rotation movement. Based on the structural form, the ranging module further includes an angle sensor, and the angle sensor is configured to measure a deflection angle of the lateral imaging probe; and method steps for the ranging module to obtain the mapping depth of focus D include:
(1) measuring a movement distance of the lateral imaging probe along the axis of the lateral imaging probe through the capacitance-grid electronic rangefinder;
(2) measuring the deflection angle through the angle sensor; and
(3) performing calculation on the distance compensation value and the deflection angle according to a geometrical relationship, and then adding a calculation result and the movement distance together to obtain the mapping depth of focus D.

Further, the temperature measurement module includes a temperature measurement processing unit, and the temperature measurement processing unit is electrically connected to the device and is configured to control the device. The temperature measurement processing unit includes an ultrasound temperature measurement method based on ultrasound radio frequency data inside. The temperature measurement processing unit calculates a mapping (projection) region of the temperature measurement ROI region within a field of view of the lateral imaging probe based on a relative geometric relationship between the focused transducer and the lateral imaging probe. The geometric relationship is determined by an actual structure design. The temperature measurement processing unit calculates an ultrasound radio frequency data acquisition boundary (hereinafter referred to as an acquisition boundary) based on the mapping region. The acquisition boundary is used for selecting a range of ultrasound radio frequency data that needs to be acquired. To ensure that the temperature measurement ROI region can obtain a complete temperature measurement distribution and higher temperature measurement precision, the acquisition boundary generally adds a certain boundary threshold based on the mapping region, and the threshold is generally set according to an actual situation.

Further, the temperature measurement processing unit transmits the mapping depth of focus and the acquisition boundary to the imaging ultrasound module.

Further, the imaging ultrasound module crops entire ultrasound radio frequency data based on the mapping depth of focus and the acquisition boundary to obtain acquisition region radio frequency data.

Further, the imaging ultrasound module transmits the acquisition region radio frequency data to the temperature measurement module. In the present invention, the reason for selecting the acquisition boundary instead of transmitting all the ultrasound radio frequency data of an entire ultrasound image to the temperature measurement module is to reduce data bandwidth pressure between the temperature measurement module and the imaging ultrasound module, thereby improving a data transmission frequency and implementing high-frame-rate ultrasound temperature measurement. In an actual embodiment, if there is sufficient bandwidth margin, data cropping may not be performed. A larger radio frequency data range is beneficial to improving the ultrasound temperature measurement precision.

Further, the position control module controls the lateral imaging probe to perform cyclic scanning on the temperature measurement ROI region, so that a plurality of groups of mapping region temperature data covering the entire ROI region may be obtained. A frequency and precision of cyclic scanning are determined according to an actual requirement.

Further, the temperature measurement processing unit constructs a temperature distribution of the entire temperature measurement ROI region based on the geometric relationship between the mapping region and the temperature measurement ROI region by using all the mapping region temperature data obtained through cyclic scanning.

Further, when a new group of mapping region temperature data is obtained, the temperature measurement processing unit quickly updates data of a corresponding position of the new group of mapping region temperature data in the temperature distribution of the temperature measurement ROI region.

It should be noted that, in an actual embodiment, for a non-phased array focused ultrasound device, when a target switches a position during treatment, relative positions of the lateral imaging probe and the focused ultrasound transducer change, and the mapping depth of focus also changes accordingly. In addition, during treatment, the lateral imaging probe needs to be attached to human skins. Affected by a physiological periodic movement such as human breathing, the mapping depth of focus also changes accordingly. Considering the two situations, in an actual embodiment:
(1) A measurement frequency of the mapping depth of focus by the ranging module should be greater than a frequency of the human physiological periodic movement.
(2) The processor should immediately transmit a mapping depth of focus obtained at a latest moment to the temperature measurement module, and a better method is to obtain the latest data through the ranging module only when the mapping depth of focus needs to be transmitted to the temperature measurement module. The method can maintain a periodic characteristic of the physiological periodic movement to a maximum extent, and can further align periodic data through methods such as respiratory gating.
(3) Another method to deal with the physiological periodic movement is: each measurement of the mapping depth of focus by the ranging module is at a same moment with the physiological periodic movement. The periodic data may be set based on actual measurement of different human bodies.

In a case, the temperature measurement ROI region is three-dimensional. In this embodiment, the mapping region is a two-dimensional region.

In another case, the temperature measurement ROI region is two-dimensional and is an axial section of a focal region of the focused ultrasound transducer, and may also include a distribution region of nearby tissues in a plane in which the axial section of the focal region is located. Since the focal region of the focused ultrasound transducer is generally an axisymmetric ellipsoid, for ease of design and calculation, in the embodiments, an axial section of a focal region perpendicular to the axis of the lateral imaging probe is generally selected as the temperature measurement ROI region. In this embodiment, the mapping region is changed from two dimensions to one dimension, that is, the temperature measurement ROI region is mapped as a line within the field of view of the lateral imaging probe.

According to another aspect, the present invention further provides a method for implementing axial focal region temperature measurement distribution detection through a lateral probe, including the following steps:
step 100: selecting a temperature measurement ROI region;
step 101: calculating a quantity c of layers that need to be scanned to completely cover the ROI region; Step 102: moving a lateral imaging probe to an n^{th} scanning layer through a position control module;
step 103: calculating a mapping region Sₙ of the n^{th} scanning layer;
step 104: calculating an acquisition boundary Aₙ of the n^{th} scanning layer;
step 105: measuring a movement distance X of the lateral imaging probe along an axis of the lateral imaging probe through a ranging module;
step 106: calculating a mapping depth of focus D;
step 107: selecting acquisition region radio frequency data RFAₙ from ultrasound radio frequency data of the lateral imaging probe based on D and Aₙ;
step 108: calculating temperature data TAₙ of the region Aₙ based on RFAₙ through a temperature measurement module;
step 109: selecting temperature data TS₁ of the region Sₙ from TAₙ;
step 110: scanning sequentially to obtain c groups of temperature data {TSᵢ};
step 111: reconstructing a three-dimensional temperature distribution {tempT(x,y,z)} based on the c groups of temperature data; and
step 112: reconstructing a temperature distribution {T(x,y,z)} of the temperature measurement ROI region based on coordinates of the temperature measurement ROI region and {tempT(x,y,z)}.

Further, the ranging module is first arranged at a zero position, and the axis of the lateral imaging probe passes through a focus of a focused ultrasound transducer, where a compensation distance from a front end surface of the lateral imaging probe to the focus is denoted as ΔD, and an angle between the axis of the lateral imaging probe and a vertical line of an axis of the focused ultrasound transducer is denoted as α;
the temperature measurement ROI region is then set as a cube symmetric with respect to the axis of the focused ultrasound transducer, where a height is denoted as H, a length is denoted as L, and a width is denoted as W; a step value of upward and downward movement scanning of the lateral imaging probe is set as Δd, and the quantity c of scanning layers is calculated according to Formula (1); in a mapping region of the temperature measurement ROI region within a field of view of the lateral imaging probe, a mapping region of each layer has a same size, where a length is L, a width is W₀, the mapping region Sₙ is denoted as Sₙ (L, W₀), and W₀ is calculated according to Formula (2):

$$c = [ \frac{H + 2 W tan α}{Δ d} ] \qquad ( 1 )$$

$${W}_{0} = \frac{W}{cos α} ; \qquad ( 2 )$$

$$and$$

the depth of focus D is calculated according to Formula (4):

$$D = X + Δ D . \qquad ( 4 )$$

Further, the ranging module is first arranged at a zero position, and the axis of the lateral imaging probe passes through a focus of a focused ultrasound transducer, where a compensation distance from a front end surface of the lateral imaging probe to the focus is denoted as ΔD, and an angle between the axis of the lateral imaging probe and a vertical line of an axis of the focused ultrasound transducer is denoted as α;
the temperature measurement ROI region is set as a cube symmetric with respect to the axis of the focused ultrasound transducer, where a height is denoted as H, a length is denoted as L, and a width is denoted as W; H₁ is a vertical distance from the focus to an upper plane of the temperature measurement ROI region, H₂ is a vertical distance from the focus to a lower plane of the temperature measurement ROI region, and a fan-shaped angle required for covering the temperature measurement ROI region is calculated by Formula (5); a step angle of fan-shaped scanning of the lateral imaging probe is set as Δ, and the quantity c of scanning layers is calculated according to Formula (6); and referring to FIG. 5, in a mapping region of the temperature measurement ROI region within a field of view of the lateral imaging probe, a mapping region of each layer has a same length L and a different width, where the width of an n^{th} layer is denoted as Wₙ, the mapping region Sₙ is denoted as Sₙ(L, Wₙ), and Wₙ is calculated according to Formula (7):

$$β = arctan ( \frac{{H}_{1}}{( Δ D + P L ) cos \frac{W}{2}} - tan α ) + arctan ( \frac{{H}_{2}}{( Δ D + P L ) cos α \frac{W}{2}} + tan α ) \qquad ( 5 )$$

in the formula, PL is a length from a front end surface to a tail end of the lateral imaging probe;

$$c = [ \frac{β}{Δ β} ] \qquad ( 6 )$$

$${W}_{n} = \frac{W}{cos ( arctan ( \frac{{H}_{2}}{( Δ D + P L ) cos α - \frac{W}{2}} + tan α ) - n Δβ )} ; \qquad ( 7 )$$

and
the depth of focus D is calculated according to Formula (8):

$$D = \frac{( Δ D + PL ) cos α}{cos ( arc tan ( \frac{{H}_{2}}{( Δ D + PL ) cos α - \frac{W}{2}} + tan α ) - n Δ β )} - PL + X . \qquad ( 8 )$$

Further, a boundary threshold added by the acquisition boundary is set as (Δa, Δb): where Δa is a threshold added in a length direction, Δb is a threshold added in a width direction, and the acquisition boundary Aₙ is calculated according to Formula (3);

$${A}_{n} = {S}_{n} + ( Δ a , Δ b ) = ( L + Δ a , {W}_{0} + Δ b ) ; \qquad ( 3 )$$

an imaging ultrasound module then selects the acquisition region radio frequency data RFAₙ from the ultrasound radio frequency data of the lateral imaging probe based on the depth of focus D and the acquisition boundary Aₙ, and transmits the acquisition region radio frequency data RFAₙ to the temperature measurement module; and the temperature measurement module calculates the temperature data TAₙ based on RFAₙ through a temperature measurement algorithm; and selects the temperature data TSₙ of the region S₁ from TAₙ according to Formula (3); and
after measurement of temperature data of a current layer is completed, the temperature measurement module adjusts the lateral imaging probe to move to an (n+1)^{th} scanning layer through the position control module, and performs the foregoing calculation step to obtain temperature data TSₙ₊₁; scanning on the c layers is completed sequentially to obtain the c groups of temperature data {TSᵢ}; the three-dimensional temperature distribution {tempT(x,y,z)} is constructed through {TSᵢ} according to the scanning interlayer step value Δd; and the temperature distribution {T(x,y,z)} of the temperature measurement ROI region is selected from {tempT(x,y,z)} based on the coordinates of the temperature measurement ROI region.

The present invention has the following beneficial effects: The apparatus and method for implementing axial focal region temperature measurement distribution detection through a lateral probe provided by the present invention can resolve the problem that an ultrasound temperature method cannot be applied since ultrasound radio frequency data is interfered with by artifacts caused by a coaxial design of an imaging ultrasound probe and a transducer, and can implement two-dimensional or three-dimensional temperature distribution detection in an axial focus region.

Additional aspects and advantages of the present invention is given in the following description, some of which becomes apparent from the following description or may be learned from practices of the present invention.

### BRIEF DESCRIPTION OF THE DRAWINGS

FIG. 1 is a schematic architectural diagram of an apparatus for implementing axial temperature measurement through a lateral probe;

In the figure: 1 is a position control module, 2 is a ranging module, 3 is a lateral imaging probe, 31 is a temperature measurement module, and 32 is an imaging ultrasound module.

FIG. 2 is an implementation flowchart of a temperature measurement method according to the present invention.

FIG. 3 is a schematic diagram of a partial structure according to an embodiment;

In the figure: 1 is a position control module, 4 is a focused ultrasound transducer, 5 is an example of an axis of the focused ultrasound transducer, 6 is an example of an axis of an lateral imaging probe, 7 is an example of a focal region of the focused ultrasound transducer, 8 is an example of a temperature measurement ROI region, 12 is an example of an angle α between the axis of the lateral imaging probe and a vertical line of the axis of the focused ultrasound transducer, 18 is an example of an initial zero position of a ranging module, and 19 is an example of a distance compensation value ΔD of the ranging module.

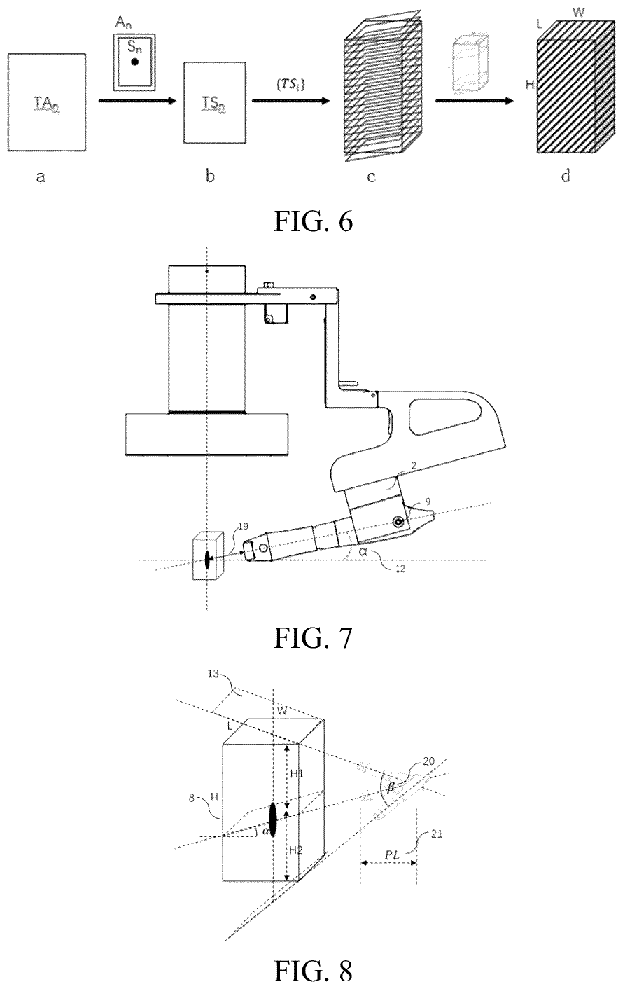

FIG. 4 is a schematic diagram of layered scanning on the temperature measurement ROI region of the embodiment shown in FIG. 3;

In the figure, 13 is an example of a mapping region.

FIG. 5 is a schematic diagram of a mapping region and an acquisition boundary, in which (a) is an example of a mapping region Sₙ of an ROI region within a field of view of a lateral imaging probe, and (b) is an example of the mapping region Sₙ and an acquisition boundary Aₙ;

In the figure: 14 is an example of the acquisition boundary, 15 is an example of a mapping focus, 16 is an example of a mapping depth of focus D, and 17 is an example of the field of view of the lateral imaging probe.

FIG. 6 is a schematic diagram of a process of reconstructing a temperature measurement distribution in a temperature measurement ROI region.

FIG. 7 is a schematic diagram of a partial structure according to another embodiment;

In the figure: 9 is a fan-shaped rotating mechanism.

FIG. 8 is a schematic diagram of layered scanning on the temperature measurement ROI region of the embodiment shown in FIG. 7; and

In the figure: 20 is a fan-shaped angle required for a lateral imaging probe to cover the temperature measurement ROI region, 21 is a length PL from a front end surface of the lateral imaging probe to a rotation shaft of a fan-shaped moving mechanical apparatus, H₁ is a vertical distance from a focus to an upper plane of the temperature measurement ROI region, and H₂ is a vertical distance from the focus to a lower plane of the temperature measurement ROI region.

### DETAILED DESCRIPTION

The following describes the present invention in detail with reference to accompanying drawings and specific embodiments.

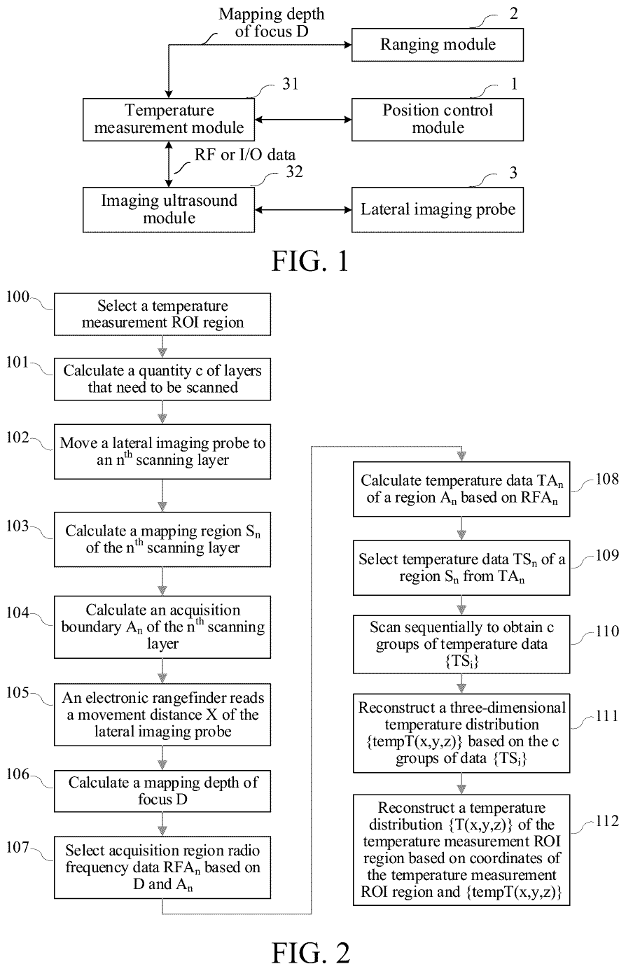

FIG. 1 shows a schematic architectural diagram of an embodiment of the present invention. In this embodiment, an apparatus provided by the present invention includes a temperature measurement module, an imaging ultrasound module, a lateral imaging probe, a ranging module, and a position control module.

The ranging module is connected to the temperature measurement module. The ranging module is configured to obtain a mapping depth of focus D of a temperature measurement ROI region within a field of view of the lateral imaging probe, and transmit the mapping depth of focus D to the temperature measurement module.

The imaging ultrasound module is connected to the temperature measurement module and the lateral imaging probe. The imaging ultrasound module sends an electrical pulse signal to the lateral imaging probe, obtains an ultrasound image and ultrasound radio frequency data through the lateral imaging probe, and transmits the ultrasound radio frequency data to the temperature measurement module. Herein, the ultrasound radio frequency data is specifically RF data.

The position control module is connected to the temperature measurement module. The temperature measurement module controls the lateral imaging probe to perform cyclic scanning on the temperature measurement ROI region through the position control module.

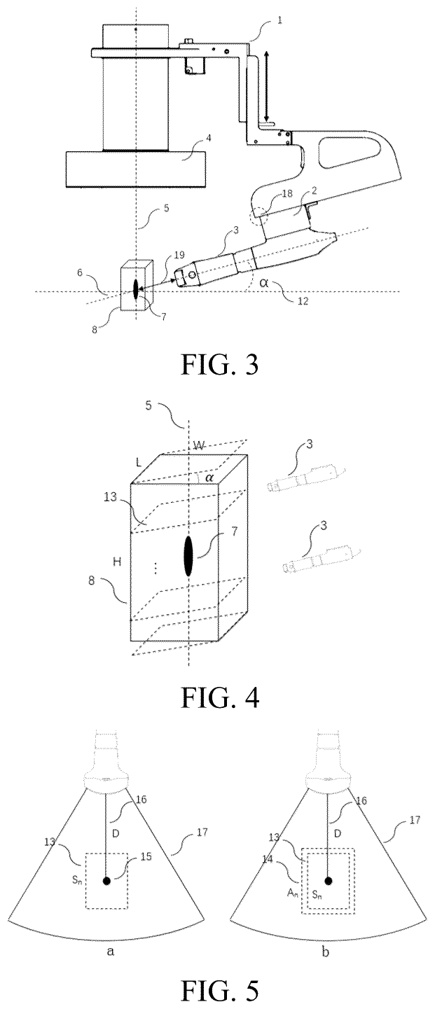

FIG. 2 shows a flowchart of a method according to an embodiment of the present invention.

Step 100. Select a temperature measurement ROI region. In an actual application, to simplify calculation, the temperature measurement ROI region is generally selected as a regular shape, for example, a cubic region symmetric with respect to an axis of a focused ultrasound transducer.

Step 101. Calculate a quantity c of layers that need to be scanned to completely cover the ROI region.

Step 102. Move a lateral imaging probe to an n^{th} scanning layer through a position control module.

Step 103. Calculate a mapping region Sₙ of the n^{th} scanning layer.

Step 104. Calculate an acquisition boundary Aₙ of the n^{th} scanning layer.

Step 105. An electronic rangefinder reads a movement distance X of the lateral imaging probe.

Step 106. Calculate a mapping depth of focus D.

Step 107. Select acquisition region radio frequency data RFAₙ from ultrasound radio frequency data of the lateral imaging probe based on D and Aₙ.

Step 108. A temperature measurement module calculates temperature data TAₙ of the region Aₙ based on RFAₙ.

Step 109. Select temperature data T_{S} of the region Sₙ from TAₙ. Step 110. Scan sequentially to obtain c groups of temperature data {TSᵢ}.

Step 111. Reconstruct a three-dimensional temperature distribution {tempT(x,y,z)} based on the c groups of temperature data.

Step 112. Reconstruct a temperature distribution {T(x,y,z)} of the temperature measurement ROI region based on coordinates of the temperature measurement ROI region and {tempT(x,y,z)}.

FIG. 3 shows an embodiment of a lateral imaging probe, a ranging module, and a position control module. In this embodiment, the lateral imaging probe is mounted on the ranging module, the ranging module is mounted on the position control module, the position control module is mounted on a focused ultrasound transducer, and an axis of the lateral imaging probe always intersects with an axis of the focused ultrasound transducer.

In this embodiment, the position control module includes a mechanical apparatus that can move in parallel upward and downward and can drive the lateral imaging probe to move in parallel in an up-down direction, and a movement direction is parallel to the axis of the focused ultrasound transducer. In this embodiment, the ranging module may move forward and backward on the position control module, and a movement direction is parallel to the axis of the lateral imaging probe. The ranging module is designed as a capacitance-grid sensor electronic rangefinder. Measurement precision may reach 0.1 mm, and a measurement frequency is 500 Hz, which is much greater than a frequency of human breathing movement. Since the lateral imaging probe is mounted on the ranging module, when the lateral imaging probe moves forward and backward along the axis of the lateral imaging probe, the ranging module may be driven to move, thereby implementing measurement of a movement distance of the lateral imaging probe.

FIG. 3 is an initial state of this embodiment. In this case, the ranging module is at a zero position, and the axis of the lateral imaging probe passes through a focus of the focused ultrasound transducer (a center of a focal region), where a compensation distance from a front end surface of the lateral imaging probe to the focus is denoted as ΔD, and an angle between the axis of the lateral imaging probe and a vertical line of the axis of the focused ultrasound transducer is denoted as α.

FIG. 4 shows scanning on a temperature measurement ROI region by the lateral imaging probe shown in FIG. 3. For ease of calculation, the temperature measurement ROI region is set as a cube symmetric with respect to the axis of the focused ultrasound transducer, where a height is denoted as H, a length is denoted as L, and a width is denoted as W. A step value of upward and downward movement scanning of the lateral imaging probe is set as Δd, and a quantity c of scanning layers is calculated according to Formula (1). With reference to FIG. 5, a dashed box in the figure is a mapping region of the temperature measurement ROI region within a field of view of the lateral imaging probe, a mapping region of each layer has a same size, where a length is L, a width is W₀, and the mapping region Sₙ is denoted as Sₙ (L, W₀). W₀ is calculated according to Formula (2).

$$c = [ \frac{H + 2 W tan α}{Δ d} ] \qquad Formula ( 1 )$$

$${W}_{0} = \frac{W}{cos α} \qquad Formula ( 2 )$$

A boundary threshold added by the acquisition boundary is set as (Δa, Δb): where Δa is a threshold added in a length direction, Δb is a threshold added in a width direction, and the acquisition boundary Aₙ is calculated according to Formula (3).

$${A}_{n} = {S}_{n} + ( Δ a , Δ b ) = ( L + Δ a , {W}_{0} + Δ b ) \qquad Formula ( 3 )$$

Referring to FIG. 3 and FIG. 5, the temperature measurement module measures a movement distance X of the lateral imaging probe through the ranging module, and calculates the depth of focus D according to Formula (4). In this embodiment, the depth of focus D is a center point of the acquisition boundary Aₙ.

$$D = X + Δ D \qquad Formula ( 4 )$$

Referring to FIG. 5(b), the imaging ultrasound module selects the acquisition region radio frequency data RFAₙ from the ultrasound radio frequency data of the lateral imaging probe based on the depth of focus D and the acquisition boundary Aₙ, and transmits the acquisition region radio frequency data RFAₙ to the temperature measurement module.

Referring to FIG. 6, the temperature measurement module calculates the temperature data TAₙ based on RFAₙ through a temperature measurement algorithm. The temperature measurement algorithm used in this embodiment is an ultrasound temperature measurement method published in patent ZL201710876349.5. According to Formula (3), the temperature data TSₙ of the region Sₙ is selected from TAₙ.

After measurement of temperature data of a current layer is completed, the temperature measurement module adjusts the lateral imaging probe to move to an (n+1)^{th} scanning layer through the position control module, and performs the foregoing calculation step to obtain temperature data TSₙ₊₁. Scanning on the c layers is completed sequentially to obtain the c groups of temperature data {TSᵢ}. Based on the scanning interlayer step value Δd, the three-dimensional temperature distribution {tempT(x, y, z)} is reconstructed through {TSᵢ}, as shown in FIG. 6(c). Based on the coordinates of the temperature measurement ROI region, the temperature distribution {T(x,y,z)} of the temperature measurement ROI region is selected from {tempT(x,y,z)}, as shown in FIG. 6(d).

FIG. 7 shows another embodiment of a lateral imaging probe, a ranging module, and a position control module. In this embodiment, a tail end of the lateral imaging probe is mounted on the position control module, the position control module is mounted on the ranging module, the ranging module is mounted on a focused ultrasound transducer through a mechanical structure, and an axis of the lateral imaging probe always intersects with an axis of the focused ultrasound transducer.

In this embodiment, the position control module includes a mechanical apparatus that can rotate in a fan shape and can drive the lateral imaging probe to move in a fan-shaped direction. The position control module is designed with an angle sensor that can measure an angle of fan-shaped rotation. The ranging module is designed as a capacitance-grid sensor electronic rangefinder. Measurement precision may reach 0.1 mm, and a measurement frequency is 500 Hz, which is much greater than a frequency of human breathing movement. Since the lateral imaging probe is mounted on the ranging module through the position control module, when the lateral imaging probe moves forward and backward along the axis of the lateral imaging probe, the ranging module may be driven to move, thereby implementing measurement of a movement distance of the lateral imaging probe.

FIG. 7 is an initial state of this embodiment. In this case, the ranging module is at a zero position, and the axis of the lateral imaging probe passes through a focus of the focused ultrasound transducer (a center of a focal region), where a compensation distance from a front end surface of the lateral imaging probe to the focus is denoted as ΔD, and an angle between the axis of the lateral imaging probe and a vertical line of the axis of the focused ultrasound transducer is denoted as α.

FIG. 8 shows scanning on a temperature measurement ROI region by the lateral imaging probe shown in FIG. 7. For ease of calculation, the temperature measurement ROI region is set as a cube symmetric with respect to the axis of the focused ultrasound transducer, where a height is denoted as H, a length is denoted as L, and a width is denoted as W. H₁ is a vertical distance from the focus to an upper plane of the temperature measurement ROI region, and H₂ is a vertical distance from the focus to a lower plane of the temperature measurement ROI region. A fan-shaped angle required for covering the temperature measurement ROI region is calculated by Formula (5). A step angle of fan-shaped scanning of the lateral imaging probe is set as Δd, and a quantity c of scanning layers is calculated according to Formula (6). Referring to FIG. 5, a dashed box in the figure is a mapping region of the temperature measurement ROI region within a field of view of the lateral imaging probe, a mapping region of each layer has a same length L and a different width, where a width of an n^{th} layer is denoted as Wₙ, and the mapping region S₁ is denoted as Sₙ (L, Wₙ). Wₙ is calculated according to Formula (7).

$$β = arc tan ( \frac{{H}_{1}}{( Δ D + PL ) cos α - \frac{W}{2}} - tan α ) + arc tan ( \frac{{H}_{2}}{( Δ D + PL ) cos α - \frac{W}{2}} + tan α ) \qquad Formula ( 5 )$$

In the formula, PL is a length from a front end surface to a tail end of the lateral imaging probe.

$$c = [ \frac{β}{Δβ} ] \qquad Formula ( 6 )$$

$${W}_{n} = \frac{W}{cos ( arc tan ( \frac{{H}_{2}}{( Δ D + PL ) cos α \frac{W}{2}} + tan α ) - n Δβ )} \qquad Formula ( 7 )$$

The depth of focus D is calculated according to Formula (8):

$$D = \frac{( Δ D + PL ) cos α}{cos ( arc tan ( \frac{{H}_{2}}{( Δ D + PL ) cos α \frac{W}{2}} + tan α ) - n Δβ )} - PL + X \qquad Formula ( 8 )$$

Subsequent temperature measurement steps of this embodiment are similar to the calculation manner of the embodiment shown in FIG. 3, and reference may be made to the foregoing content.

The basic principles, main features, and advantages of the present invention are shown and described above. A person of ordinary skill in the art should understand that the foregoing embodiments are not intended to limit the protection scope of the present invention in any form, and all technical solutions obtained by manners such as equivalent replacement fall within the protection scope of the present invention.

All parts not involved in the present invention are the same as the related art or may be implemented by using the related art.

1. An apparatus for implementing axial focal region temperature measurement distribution detection through a lateral probe, comprising a temperature measurement module, an imaging ultrasound module, a lateral imaging probe, a ranging module, and a position control module, wherein
the imaging ultrasound module is connected to the temperature measurement module and the lateral imaging probe, and the imaging ultrasound module sends an electrical pulse signal to the lateral imaging probe, and obtains an ultrasound image and ultrasound radio frequency data through the lateral imaging probe;
the lateral imaging probe is mounted on the position control module, an axis of the lateral imaging probe always intersects with an axis of a focused ultrasound transducer, and the position control module drives the lateral imaging probe to move, to cause an imaging field of view of the lateral imaging probe to traverse a temperature measurement ROI region through movement;
the position control module is connected to the temperature measurement module, and the temperature measurement module controls the lateral imaging probe to perform cyclic scanning on the temperature measurement ROI region through the position control module; and
the ranging module is connected to the temperature measurement module, and the ranging module is configured to obtain a mapping depth of focus D of the temperature measurement ROI region within the field of view of the lateral imaging probe, and transmit the mapping depth of focus D to the temperature measurement module.

2. The apparatus for implementing axial focal region temperature measurement distribution detection through a lateral probe according to claim 1, wherein the ranging module comprises a ranging processing unit and a capacitance-grid electronic rangefinder, and is configured to measure a distance from an intersection point of the axis of the focused ultrasound transducer and the axis of the lateral imaging probe to a foremost end surface of the lateral imaging probe, that is, the mapping depth of focus D, a distance compensation value is stored inside the ranging processor unit, and the distance compensation value is a distance between the axis of the focused ultrasound transducer and a front end surface of the lateral imaging probe when the lateral imaging probe is in an initial state.

3. The apparatus for implementing axial focal region temperature measurement distribution detection through a lateral probe according to claim 2, wherein the position control module comprises a parallel moving mechanism, and the parallel moving mechanism drives the lateral imaging probe to move upward and downward in an axis direction of the focused ultrasound transducer through a bracket.

4. The apparatus for implementing axial focal region temperature measurement distribution detection through a lateral probe according to claim 2, wherein the position control module comprises a fan-shaped rotating mechanism, and the fan-shaped rotating mechanism is arranged at a tail end of a bracket on which the lateral imaging probe is mounted, and drives a front end of the lateral imaging probe to perform fan-shaped rotation movement.

5. The apparatus for implementing axial focal region temperature measurement distribution detection through a lateral probe according to claim 3, wherein method steps for the ranging module to obtain the mapping depth of focus D comprise:
(1) measuring a movement distance of the lateral imaging probe along the axis of the lateral imaging probe through the capacitance-grid electronic rangefinder; and
(2) adding the movement distance and the distance compensation value together to obtain the mapping depth of focus D.

6. The apparatus for implementing axial focal region temperature measurement distribution detection through a lateral probe according to claim 4, wherein the ranging module further comprises an angle sensor, and the angle sensor is configured to measure a deflection angle of the lateral imaging probe; and method steps for the ranging module to obtain the mapping depth of focus D comprise:
(1) measuring a movement distance of the lateral imaging probe along the axis of the lateral imaging probe through the capacitance-grid electronic rangefinder;
(2) measuring the deflection angle through the angle sensor; and
(3) performing calculation on the distance compensation value and the deflection angle according to a geometrical relationship, and then adding a calculation result and the movement distance together to obtain the mapping depth of focus D.

7. A method for implementing axial focal region temperature measurement distribution detection through a lateral probe, comprising the following steps:
step 100: selecting a temperature measurement ROI region;
step 101: calculating a quantity c of layers that need to be scanned to completely cover the ROI region;
Step 102: moving a lateral imaging probe to an n^{th} scanning layer through a position control module;
step 103: calculating a mapping region Sₙ of the n^{th} scanning layer;
step 104: calculating an acquisition boundary Aₙ of the n^{th} scanning layer;
step 105: measuring a movement distance X of the lateral imaging probe along an axis of the lateral imaging probe through a ranging module;
step 106: calculating a mapping depth of focus D;
step 107: selecting acquisition region radio frequency data RFAₙ from ultrasound radio frequency data of the lateral imaging probe based on D and Aₙ;
step 108: calculating temperature data TAₙ of the region Aₙ based on RFAₙ through a temperature measurement module;
step 109: selecting temperature data TSₙ of the region Sₙ from TAₙ;
step 110: scanning sequentially to obtain c groups of temperature data {TSᵢ};
step 111: reconstructing a three-dimensional temperature distribution {tempT(x,y,z)} based on the c groups of temperature data; and
step 112: reconstructing a temperature distribution {T(x,y,z)} of the temperature measurement ROI region based on coordinates of the temperature measurement ROI region and {tempT(x,y,z)}.

8. The method for implementing axial focal region temperature measurement distribution detection through a lateral probe according to claim 7, wherein the ranging module is first arranged at a zero position, and the axis of the lateral imaging probe passes through a focus of a focused ultrasound transducer, wherein a compensation distance from a front end surface of the lateral imaging probe to the focus is denoted as ΔD, and an angle between the axis of the lateral imaging probe and a vertical line of an axis of the focused ultrasound transducer is denoted as α;
the temperature measurement ROI region is then set as a cube symmetric with respect to the axis of the focused ultrasound transducer, wherein a height is denoted as H, a length is denoted as L, and a width is denoted as W; a step value of upward and downward movement scanning of the lateral imaging probe is set as Δd, and the quantity c of scanning layers is calculated according to Formula (1); in a mapping region of the temperature measurement ROI region within a field of view of the lateral imaging probe, a mapping region of each layer has a same size, wherein a length is L, a width is W₀, the mapping region Sₙ is denoted as Sₙ (L, W₀), and W₀ is calculated according to Formula (2):
$$c = [ \frac{H + 2 W tan α}{Δ d} ] \qquad ( 1 )$$
$${W}_{0} = \frac{W}{cos α} ; \qquad ( 2 )$$
the depth of focus D is calculated according to Formula (4):
$$D = X + Δ D . \qquad ( 4 )$$

9. The method for implementing axial focal region temperature measurement distribution detection through a lateral probe according to claim 7, wherein the ranging module is first arranged at a zero position, and the axis of the lateral imaging probe passes through a focus of a focused ultrasound transducer, wherein a compensation distance from a front end surface of the lateral imaging probe to the focus is denoted as ΔD, and an angle between the axis of the lateral imaging probe and a vertical line of an axis of the focused ultrasound transducer is denoted as α;
the temperature measurement ROI region is set as a cube symmetric with respect to the axis of the focused ultrasound transducer, wherein a height is denoted as H, a length is denoted as L, and a width is denoted as W; H₁ is a vertical distance from the focus to an upper plane of the temperature measurement ROI region, H₂ is a vertical distance from the focus to a lower plane of the temperature measurement ROI region, and a fan-shaped angle required for covering the temperature measurement ROI region is calculated by Formula (5); a step angle of fan-shaped scanning of the lateral imaging probe is set as Δ, and the quantity c of scanning layers is calculated according to Formula (6); and referring to FIG. 5, in a mapping region of the temperature measurement ROI region within a field of view of the lateral imaging probe, a mapping region of each layer has a same length L and a different width, wherein the width of an n^{th} layer is denoted as Wₙ, the mapping region Sₙ is denoted as Sₙ(L, Wₙ), and Wₙ is calculated according to Formula (7):
$$β = arc tan ( \frac{{H}_{1}}{( Δ D + PL ) cos α - \frac{W}{2}} - tan α ) + arc tan ( \frac{{H}_{2}}{( Δ D + PL ) cos α - \frac{W}{2}} + tan α ) \qquad ( 5 )$$
in the formula, PL is a length from a front end surface to a tail end of the lateral imaging probe;
$$c = [ \frac{β}{Δβ} ] \qquad ( 6 )$$
$${W}_{n} = \frac{W}{cos ( arc tan ( \frac{{H}_{2}}{( Δ D + PL ) cos α - \frac{W}{2}} + tan α ) - n Δβ )} ; \qquad ( 7 )$$
the depth of focus D is calculated according to Formula (8):
$$D = \frac{( Δ D + PL ) cos α}{cos ( arc tan ( \frac{{H}_{2}}{( Δ D + PL ) cos α - \frac{W}{2}} + tan α ) - n Δβ )} - PL + X . \qquad ( 8 )$$

10. The method for implementing axial focal region temperature measurement distribution detection through a lateral probe according to claim 8, wherein a boundary threshold added by the acquisition boundary is set as (Δa, Δb): wherein Δa is a threshold added in a length direction, Δb is a threshold added in a width direction, and the acquisition boundary Aₙ is calculated according to Formula (3);
$${A}_{n} = {S}_{n} + ( Δ a , Δ b ) = ( L + Δ a , {W}_{0} + Δ b ) ; \qquad ( 3 )$$
an imaging ultrasound module then selects the acquisition region radio frequency data RFAₙ from the ultrasound radio frequency data of the lateral imaging probe based on the depth of focus D and the acquisition boundary Aₙ, and transmits the acquisition region radio frequency data RFAₙ to the temperature measurement module; and the temperature measurement module calculates the temperature data TAₙ based on RFAₙ through a temperature measurement algorithm; and selects the temperature data TSₙ of the region Sₙ from TAₙ according to Formula (3); and
after measurement of temperature data of a current layer is completed, the temperature measurement module adjusts the lateral imaging probe to move to an (n+1)^{th} scanning layer through the position control module, and performs the foregoing calculation step to obtain temperature data TSₙ₊₁; scanning on the c layers is completed sequentially to obtain the c groups of temperature data {TSᵢ}; the three-dimensional temperature distribution {tempT(x,y,z)} is constructed through {TSᵢ} according to the scanning interlayer step value Δd; and the temperature distribution {T(x,y,z)} of the temperature measurement ROI region is selected from {tempT(x,y,z)} based on the coordinates of the temperature measurement ROI region.

11. The method for implementing axial focal region temperature measurement distribution detection through a lateral probe according to claim 9, wherein a boundary threshold added by the acquisition boundary is set as (Δa, Δb): wherein Δa is a threshold added in a length direction, Δb is a threshold added in a width direction, and the acquisition boundary Aₙ is calculated according to Formula (3);
$${A}_{n} = {S}_{n} + ( Δ a , Δ b ) = ( L + Δ a , {W}_{0} + Δ b ) ; \qquad ( 3 )$$
an imaging ultrasound module then selects the acquisition region radio frequency data RFAₙ from the ultrasound radio frequency data of the lateral imaging probe based on the depth of focus D and the acquisition boundary Aₙ, and transmits the acquisition region radio frequency data RFAₙ to the temperature measurement module; and the temperature measurement module calculates the temperature data TAₙ based on RFAₙ through a temperature measurement algorithm; and selects the temperature data TSₙ of the region Sₙ from TA₁ according to Formula (3); and
after measurement of temperature data of a current layer is completed, the temperature measurement module adjusts the lateral imaging probe to move to an (n+1)^{th} scanning layer through the position control module, and performs the foregoing calculation step to obtain temperature data TSₙ₊₁; scanning on the c layers is completed sequentially to obtain the c groups of temperature data {TSᵢ}; the three-dimensional temperature distribution {tempT(x,y,z)} is constructed through {TSᵢ} according to the scanning interlayer step value Δd; and the temperature distribution {T(x,y,z)} of the temperature measurement ROI region is selected from {tempT(x,y,z)} based on the coordinates of the temperature measurement ROI region.
